(19) United States
(12) Patent Application Publication
(10) Pub. No.: US 2026/0326008 A1
(43) Pub. Date: Oct. 22, 2026

(54) AIR FILTERING AND TREATMENT APPARATUS AND SYSTEM

(71) Applicant: VBreathe PTY LTD, Chatswood (AU)

(72) Inventors: Angus Emerson Mabin; Anthony Elio Leo, Chatswood (AU); Hans Christer Henric Engstrom, Chatswood (AU); Jingkui Li, Chatswood (AU); Lachlan Charles Chow, Chatswood (AU); Noah Sean Kelly, Chatswood (AU); Simon Nicholas Barnacoat, Chatswood (AU)

(73) Assignee: VBreathe PTY LTD, Chatswood (AU)

(21) Appl. No.: 19/104,650

(22) PCT Filed: Aug. 18, 2023
(86) PCT No.: PCT/AU2023/050784

§ 371 Date: Feb. 18, 2025

(30) Foreign Application Priority Data
Aug. 18, 2022 (AU) 2022902363

(51) Int. Cl. B01D 46/00; B01D 46/44; B01D 46/46; F24F 8/24

(52) U.S. Cl. CPC B01D 46/0028; B01D 46/0041; B01D 46/0049; B01D 46/442; B01D 46/46; F24F 8/24; B01D 2279/40

(57) ABSTRACT

An air filtering and treatment apparatus 10 including: a housing 12 having an air inlet structure 24 configured to receive ambient air, an air outlet structure 26 configured to release a first portion of filtered air 29b, and an air passageway 28 between the air inlet structure 26 and the air outlet structure 28; a fan 14 arranged to urge air through the air passageway 28 between the air inlet structure 24 and the air outlet structure 26; a controller 22 configured to control the fan 14; a filter 16 arranged to filter air passing through the air passageway 28; a receptacle 18 located toward the air outlet structure 26 relative to the filter 16 having located therein a treatment composition 56, wherein an evaporable vapour 58 associated with the treatment composition 56 is able to accumulate in a headspace 34 of the receptacle 18; and an entrainment structure 20, in fluid communication with the air passageway 28, having one or more entrainment outlets 30 for releasing a second portion 29b of filtered air 28, wherein the entrainment structure 20 is selectively movable relative to the housing 12 to control an amount of the evaporable vapour 58 entrained with the second portion 29b of filtered air 28 exiting the one or more entrainment outlets 30.

### TECHNICAL FIELD

The invention relates to an apparatus and system for air cleaning, and in particular, filtering and treatment of indoor air.

### BACKGROUND

Air quality, in particular indoor air quality, may be compromised by the presence of dust, pet dander, odours, airborne organic matter such as mould and airborne pathogens.

Accordingly, various types of indoor air treatment or filtering devices have been proposed.

One such air-filtering device includes a housing having a fan that draws air through the rear centre of the unit, passes the air through a filter and then discharges the air through a grille at the top of the housing. The filter may be a HEPA (High Efficiency Particulate Air) filter that is arranged to collect microscopic particles such as allergy provoking pollen grains, dust mites, mould spores and pet dander particles. An ioniser may also be included.

Some indoor air treatment or filtering devices may include an aromatic or disinfectant substance that is emitted from the device into the air stream. However, such devices generally emit a pre-determined or constant amount of the substance. In some instances, users have resorted to switching such devices on and off to try and vary the amount of the substance emitted. However, it is necessary for the user to remember to switch the device on and off which can be overlooked, thereby leading to the device either operating in manner where too little substance is being emitted to effectively clean the air or too much substance is being emitted resulting in an inefficient use of the substance.

### SUMMARY

The present invention seeks to overcome or at least ameliorate one or more of the above identified problems or at least provide a useful alternative.

In a first aspect there is provided an air filtering and treatment apparatus including: a housing having an air inlet structure configured to receive ambient air, an air outlet structure configured to release a first portion of filtered air, and an air passageway between the air inlet structure and the air outlet structure; a fan arranged to urge air through the air passageway between the air inlet structure and the air outlet structure; a controller configured to control the fan; a filter arranged to filter air passing through the air passageway; a receptacle located toward the air outlet structure relative to the filter having located therein a treatment composition, wherein an evaporable vapour associated with the treatment composition is able to accumulate in a headspace of the receptacle; and an entrainment structure, in fluid communication with the air passageway, having one or more entrainment outlets for releasing a second portion of filtered air, wherein the entrainment structure is selectively movable relative to the housing to control an amount of the evaporable vapour entrained with the second portion of filtered air exiting the one or more entrainment outlets.

In certain embodiments, the housing includes a vent structure which is coupled and rotatable relative to the entrainment structure, wherein the entrainment structure is configured to rotate relative to the vent structure to adjust an amount of the second portion of the filtered air entering the headspace to thereby control the amount of the evaporable vapour entrained with the second portion of filtered air exiting the one or more entrainment outlets.

In certain embodiments, the vent structure includes a plurality of inlet apertures in fluid communication with the headspace which enable the second portion of filtered air to be directed within the headspace, wherein the entrainment structure includes a wall having one or more gaps, wherein the entrainment structure is configured to rotate relative to the vent structure to adjust alignment of the one or more gaps with the plurality of inlet apertures to thereby control an amount of the evaporable vapour entrained with the second portion of filtered air exiting the one or more entrainment outlets.

In certain embodiments, the vent structure includes a channel housing a biasing component therein and a plurality of stepped recesses, wherein a neck portion of the entrainment structure has a protrusion which is receivable within, movable along and biased, by the biasing component, into engagement with one of the stepped recesses, wherein a force applied by the user to the entrainment structure along a longitudinal axis of the apparatus disengages the protrusion from a respective stepped recess thereby allowing the entrainment structure to be rotated relative the housing and allow the protrusion to engage with a different stepped recess to thereby control the amount of the evaporable vapour entrained with the second portion of the filtered air exiting the one or more entrainment outlets.

In certain embodiments, each inlet aperture is diametrically offset relative to a remainder of the inlet apertures.

In certain embodiments, the entrainment structure is configured to substantially enclose the receptacle within the housing, wherein the entrainment structure is detachable from the vent structure of the housing to allow access to the receptacle.

In certain embodiments, the air filtering and treatment apparatus further includes a position sensor, in communication with the controller, configured to sense a position of the entrainment structure relative to the housing, wherein the controller is configured to receive a position signal from the position sensor and facilitate output of the position of the entrainment assembly to the user.

In certain embodiments, the position sensor includes one or more photodetectors, wherein the air filtering and treatment apparatus includes one or more emitters, wherein the one or more photodetectors are configured to sense the presence or absence of light emitted by one or more emitters to detect a position of the entrainment assembly.

In certain embodiments, the one or more photodetectors are infrared photodetectors, and the one or more emitters are infrared emitters.

In certain embodiments, the air outlet structure includes a plurality of vanes, wherein at least some of the vanes have extending therefrom a deflecting member to deflect the second portion of the filtered air radially inward within the headspace of the receptacle such that the evaporable vapour is entrained with the second portion of filtered air when exiting the one or more entrainment outlets.

In certain embodiments, each deflecting member extends substantially orthogonally from the respective vane.

In certain embodiments, the air filtering and treatment apparatus further includes a force sensor in communication with the controller, wherein the force sensor is configured to detect a presence of a container containing the treatment composition within the receptacle and generate a force signal indicative of the presence of the container within the receptacle, wherein the controller is configured to operate the fan in response to receiving the force signal indicative of the presence of the container within the receptacle.

In certain embodiments, the force signal is indicative of an amount of force sensed by the force sensor, wherein the controller is configured to determine, based on the amount of force sensed by the force sensor, an amount of treatment composition remaining in the container and facilitate output, to the user, the amount of treatment composition remaining in the container.

In certain embodiments, the air filtering and treatment apparatus further an integrated sensing module including a fan and particle sensor, wherein the particle sensor is configured to sense a particle count of the ambient air drawn through the integrated sensing module by the fan and facilitate output of the particle count to the user.

In certain embodiments, the housing includes a door movable between an open position and closed position to access, locate and replace the filter.

In certain embodiments, the housing has a base having a convex surface that is at least partially locatable within a recess arranged at an end of the filter to locate the filter within the housing.

In certain embodiments, the air filtering and treatment apparatus further includes an input button to operate the apparatus according to a customised setting, wherein the controller is configured to: receive customised setting data indicative of a customised setting for operating the apparatus; store, in memory, an association between the customised setting and the button; receive a signal indicative of the button being selected by the user; and operate the fan according to the customised setting stored in the memory.

In another aspect, there is provided a system including: an air filtering and treatment apparatus configured according to one or more of the above embodiments including a position sensor, wherein the air filtering and treatment apparatus further includes a communication module; and a computer program executable by a processor of a remote computing device, wherein execution of the computer program by the processor configures the processor to: receive, from the communication module of the air filtering and treatment apparatus, position sensor data indicative of the position of the entrainment structure of the air filtering and treatment apparatus; and control an output device of the remote computing device to indicate to a user the sensed position of the entrainment assembly.

In another aspect there is provided a system including: an air filtering and treatment apparatus configured according to one or more of the above embodiments including a force sensor, wherein the air filtering and treatment apparatus further includes a communication module; and a computer program executable by a processor of a remote computing device, wherein execution of the computer program by the processor configures the processor to: receive, from the communication module, force sensor data indicative of the force sensed by the force sensor; and control an output device of the remote computing device to indicate to the user the amount of treatment composition remaining in the container.

In another aspect there is provided a system including: an air filtering and treatment apparatus configured according to one ore of the above embodiments including a customised setting; and a computer program executable by a processor of a remote computing device, wherein execution of the computer program by the processor configures the processor to: receive customised setting data from the user via the remote computing device; and transfer, to the communication module of the air filtering and treatment apparatus, the customised setting data for storing in memory in association with the button.

Other aspects and embodiments will be appreciated throughout the detailed description.

### BRIEF DESCRIPTION OF THE FIGURES

The invention is described, by way of non-limiting example only, by reference to the accompanying figures.

FIG. 1 is a front, top and right-side isometric view illustrating an example of the apparatus.

FIG. 2 is a front, bottom and right-side isometric view illustrating an example of the apparatus.

FIG. 3 is a front view illustrating the apparatus.

FIG. 4 is a rear view illustrating the apparatus.

FIGS. 5A and 5B are an exploded front, top and right-side isometric view Illustrating the apparatus.

FIG. 6 is a front sectional view illustrating the apparatus.

FIG. 7 is a side sectional view illustrating the apparatus.

FIG. 8 is a top and front view of an entrainment structure and the vent structure of the apparatus.

FIG. 9 is a top and left side view of the entrainment structure, the vent structure, the container, and a position sensor.

FIG. 10 is an underside view of the entrainment structure, the vent structure, the container, the position sensor and a plunger.

FIG. 11 is an underside view of the entrainment structure and vent structure.

FIG. 12 is an underside view of a lid portion of the entrainment structure.

FIG. 13 is an underside view of a vent structure of the housing.

FIG. 14 is a top view of the vent structure of the housing,

FIG. 15 is a magnified underside view of vanes of the vent structure.

FIG. 16 is an exploded top and side view of the container, the plunger, a force sensor and associated circuit board.

FIG. 17 is an exploded bottom and side view of the container, the plunger, a force sensor and associated circuit board.

FIG. 18 is a magnified, front sectional view of the container supported upon the plunger pressing against the force sensor.

FIG. 19 is a rear, bottom, isometric view of the apparatus with the door in the open position.

FIG. 20 is a rear, top, isometric view of the apparatus with the door in the open position.

FIG. 21a a side view of a head cap of the filter of the apparatus.

FIG. 21b is a side view of a base cap of the filter of the apparatus.

FIG. 22 is a side view of the base of the apparatus.

FIG. 23 is a top and side view of a filter of the apparatus.

FIG. 24 is an underside and side view of the filter of the apparatus.

FIG. 25 is a functional block diagram of an example of a control system of the apparatus.

FIG. 26 is a system diagram showing a system including the apparatus, a server processing system, and a remote communication device.

FIG. 27 is a functional block diagram of a remote computing device.

### DETAILED DESCRIPTION

The following modes, given by way of example only, are described to provide a more precise understanding of the subject matter of a preferred embodiment or embodiments. In the figures, incorporated to illustrate features of an example embodiment, like reference numerals are used to identify like parts throughout the figures.

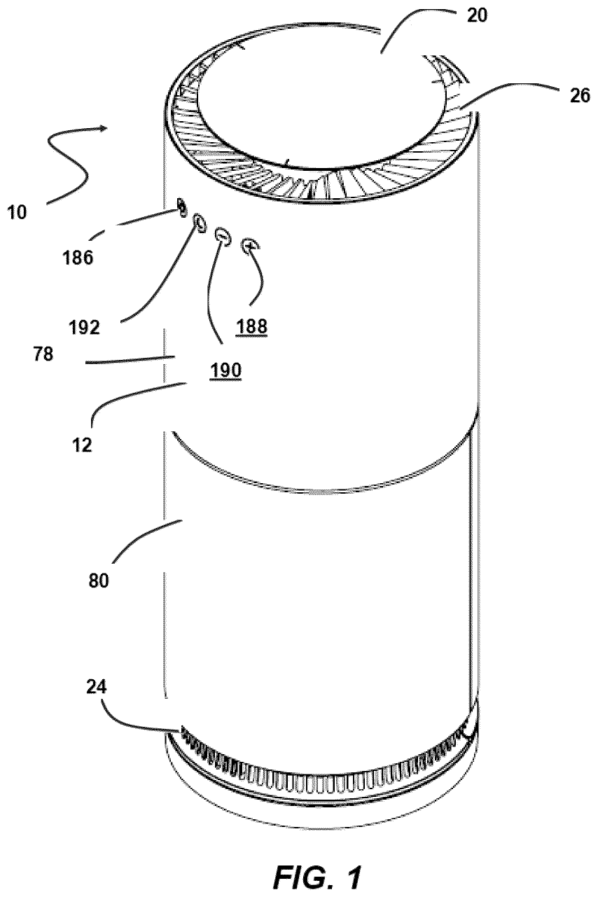

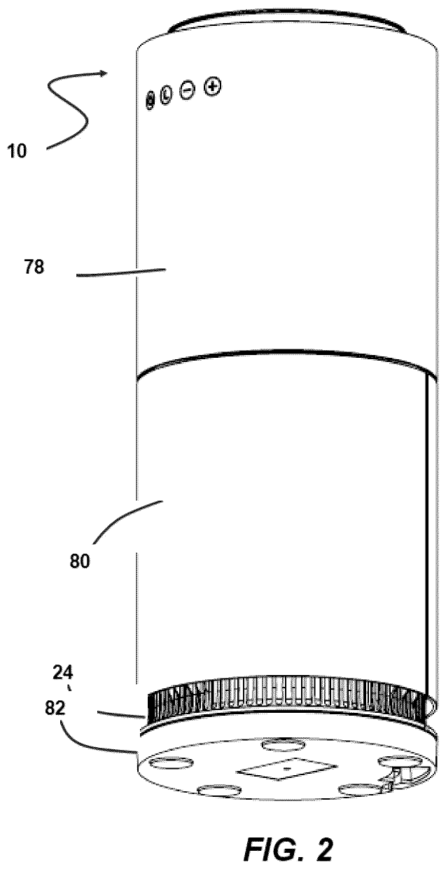

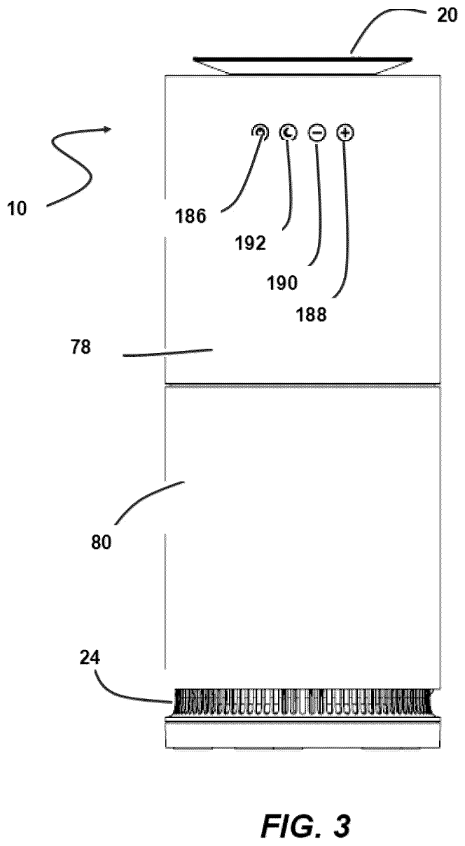

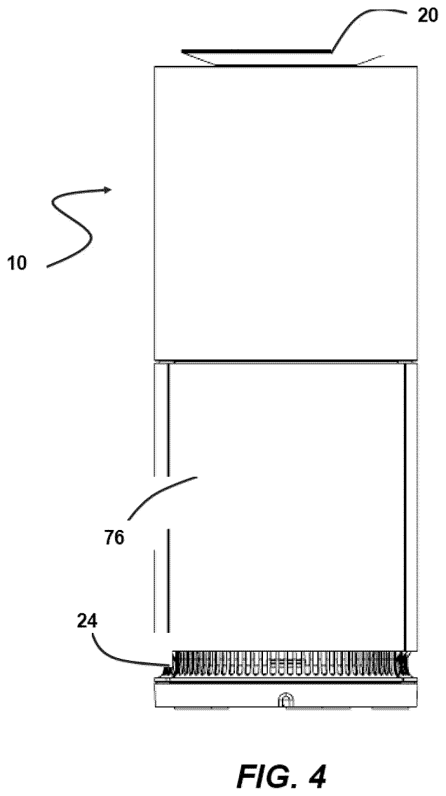

Referring to FIGS. 1 to 7, there is shown schematics of an example of an air filtering and treatment apparatus 10. The apparatus 10 is configured to filter 16 and treat indoor air. The air filtering and treatment apparatus 10 includes a housing 12, a fan 14, a filter 16, a receptacle 18, an entrainment structure 20, and a controller 22. The housing 12 has an air inlet structure 24 configured to receive ambient air. The housing 12 further includes an air outlet structure 26 configured to release a first portion 29a of filtered air. The housing 12 further includes an air passageway 28 arranged between the air inlet structure 24 and the air outlet structure 26. The filter 16 is arranged to filter air passing through the air passageway 28. The fan 14 is arranged to urge air through the air passageway 28 between the air inlet structure 24 and the air outlet structure 26. The controller 22 is configured to control the fan 14 to urge the air to be drawn within the air inlet structure 24 and exit the air outlet structure 26. The receptacle 18 is located toward the air outlet structure 26 relative to the filter 16 having located therein a treatment composition 56, wherein an evaporable vapour 58 associated with the treatment composition 56 is able to accumulate in a headspace 34 of the receptacle 18. The entrainment structure 20 is in fluid communication with the air passageway 28, having one or more entrainment outlets 30 for releasing a second portion 29b of filtered air. The entrainment structure 20 is selectively movable relative to the housing 12 to control an amount of the evaporable vapour 58 entrained with the second portion of filtered air exiting the one or more entrainment outlets 30.

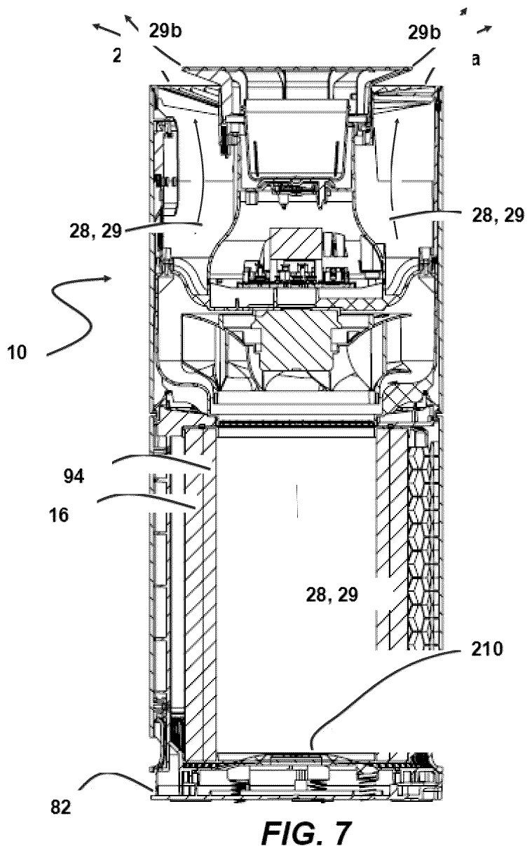

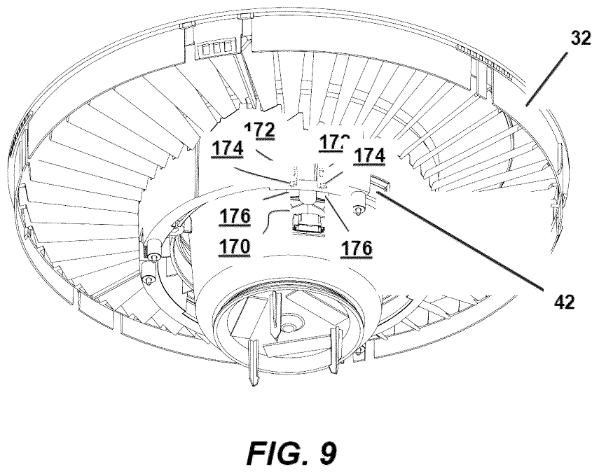

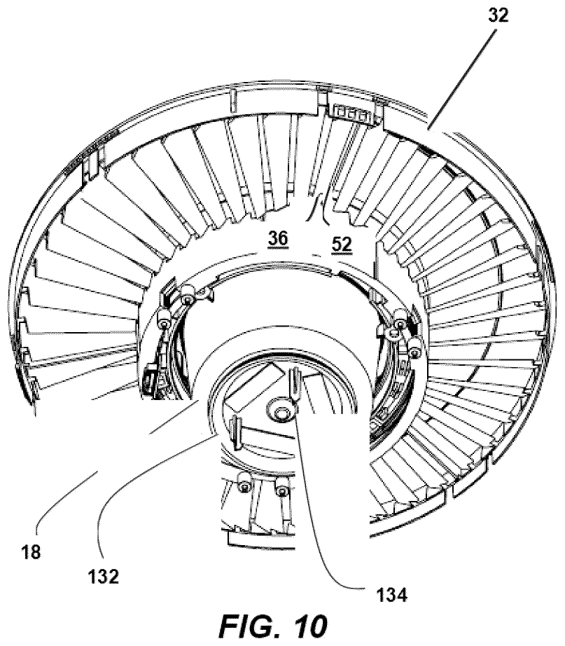

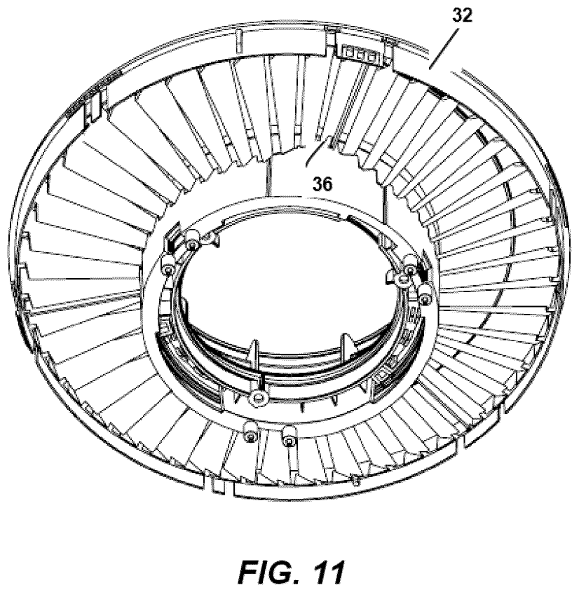

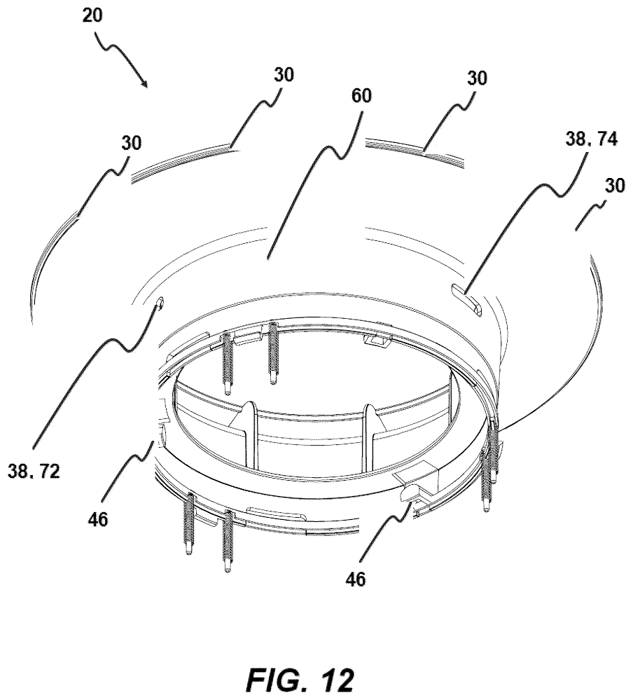

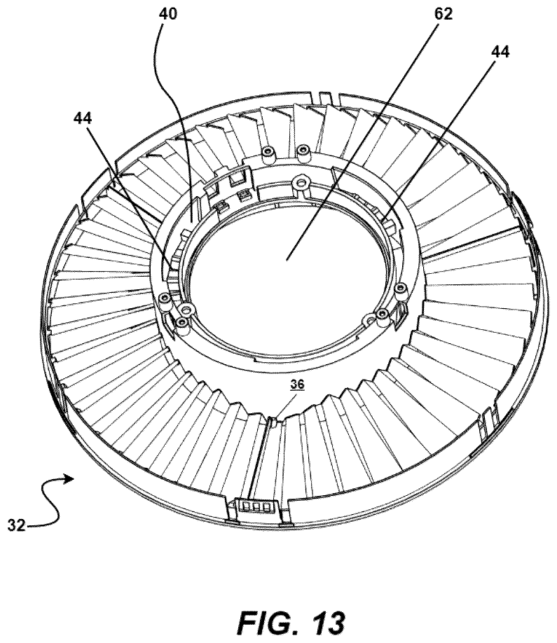

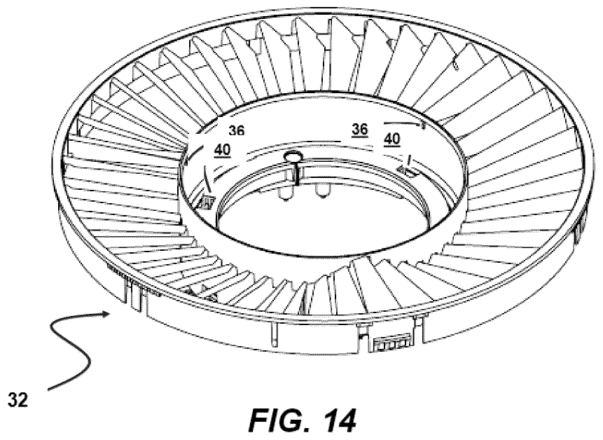

More specifically, referring to FIGS. 8 to 15, the housing 12 includes a vent structure 32 which is coupled and rotatable relative to the entrainment structure 20. The entrainment structure 20 is configured to rotate relative to the vent structure 32 to adjust an amount of the second portion of the filtered air entering the headspace 34 to thereby control the amount of the evaporable vapour 58 entrained with the second portion of filtered air exiting the one or more entrainment outlets 30.

The vent structure 32 includes a plurality of inlet apertures 36 in fluid communication with the headspace 34 of the receptacle 18 which enable the second portion of filtered air to be directed within the headspace 34. The entrainment structure 20 includes a wall 60 having one or more entrainment inlets 38, wherein the entrainment structure 20 is configured to rotate relative to the vent structure 32 to adjust alignment of the one or more inlet apertures 36 with the one or more entrainment inlets 38 to thereby control an amount of the evaporable vapour 58 entrained with the second portion of filtered air exiting the one or more entrainment outlets 30. Each inlet aperture 36 is diametrically offset relative to a remainder of the inlet apertures 36. Similarly, each entrainment inlet 38 in the wall of the entrainment structure 20 is diametrically offset relative to the remainder of the entrainment inlets 38 in the wall. The diametric offsetting of the inlet apertures 36 and the entrainment inlets 38 advantageously avoids direct interference of opposing incoming streams of filtered air within the headspace 34 of the receptacle 18.

The vent structure 32 also includes a channel 40 housing a biasing component 42 therein and a plurality of stepped recesses 44. A neck portion of the entrainment structure 20 has a protrusion 46 which is receivable within and movable along the channel 40 as well as being biased, by the biasing component 42 provided in the form of a spring, into engagement with one of the stepped recesses 44 located along the channel 40. A force applied by the user to the entrainment structure 20 along a longitudinal axis 48 of the apparatus 10 disengages the protrusion 46 from a respective stepped recess 44 thereby allowing the entrainment structure 20 to be rotated relative the housing 12 such that the protrusion 46 can be located to engage with a different stepped recess 44 once the downward force is released to thereby control the amount of the evaporable vapour 58 entrained with the second portion of the filtered air exiting the one or more entrainment outlets 30.

The air outlet structure 26 of the housing 12, provided in the form of the vent structure 32, includes a plurality of vanes 50. At least some of the vanes 50 have extending therefrom a deflecting member 52 to deflect the second portion of the filtered air radially inward within the headspace 34 of the receptacle 18 such that the evaporable vapour 58 is entrained with the second portion of filtered air when exiting the one or more entrainment outlets 30. In one form, each deflecting member 52 extends substantially orthogonally from the respective vane 50.

The entrainment structure 20 is configured to substantially enclose the receptacle 18 within the housing 12. The entrainment structure 20 is detachable from the vent structure 32 of the housing 12 to allow access to the receptacle 18. The entrainment structure 20 together with the receptacle 18 define the headspace 34 above the treatment composition 56 contained in a container 54.

The entrainment structure 20 has a first portion 62 having a planar profile that is secured to a shouldered end of a second portion 64. The first portion 62 is preferably ultrasonically welded to the shouldered end of the second portion 64. An upper edge of the shouldered end includes the entrainment outlets 30. The underside surface of the flared end of the second portion 64 is tapered to deflect filtered air flowing out of the vent structure 32 outwardly and laterally. The tapered underside surface of the second portion 64 of the entrainment structure 20 also provides a clearance to allow a user to place their hands under the entrainment structure 20 to lift the apparatus 10. An inner surface of the wall defining the second portion 64 of the entrainment structure 20 includes a plurality of “L-shaped” ribs 68 which provide support to the strength of the entrainment structure 20 as well as provide support for the planar underside surface of the first portion 62 of the entrainment structure 20.

The wall 60 defining a downwardly extending neck 70 of the second portion 64 includes the entrainment inlets 38. The entrainment inlets 38 are located at the upper portion of the neck 70 adjacent the lower portion of the shouldered section. In particular, the entrainment inlets 38 include a first and second entrainment inlet, 72, 74. The first and second inlet 72, 74 are preferably of differing sizes. The first inlet 72 is generally a hole, and the second inlet 74 is generally a slot. When the entrainment structure 20 is moved to a maximum entrainment position, inlet apertures 36 of the vent structure 32 align with both the first and second entrainment inlets 72, 74, thereby allowing filtered air to enter the headspace 34 through both the first and second entrainment inlets 72, 74. In an intermediate entrainment position, the wall 60 of the vent structure 32 blocks or covers the first entrainment inlet 72. However, the second entrainment inlet 74 is a slot which one of the inlet apertures 36 of the vent structure 32 moves along such that filtered air continues to flow into the second entrainment inlet 74. In the minimum entrainment position, the wall 60 of the vent structure 32 blocks or covers the first entrainment inlet 72 and the second entrainment inlet 74 such that filtered air substantially does not flow into the headspace 34 of the receptacle 18.

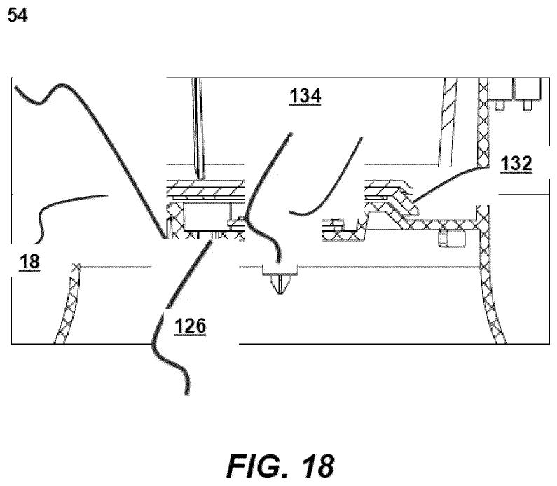

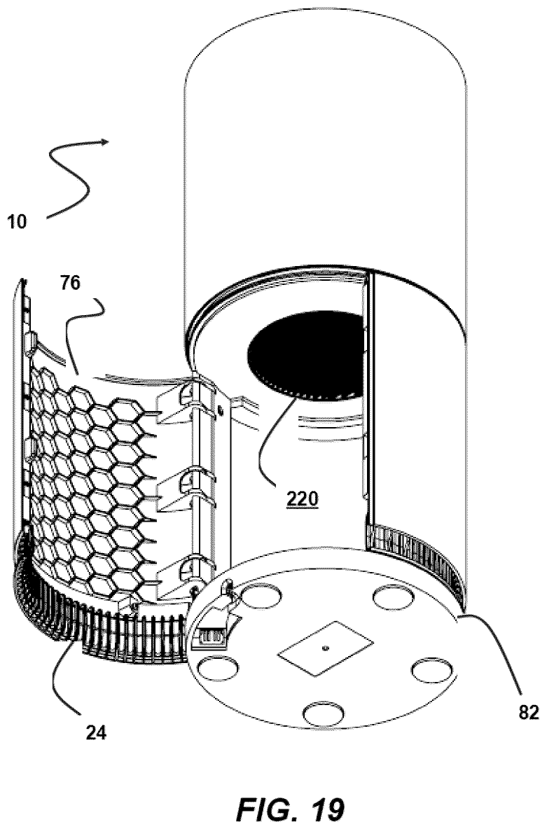

Referring to FIGS. 19 and 20, the housing 12 preferably includes a door 76 movable between an open position and closed position to access, locate and replace the filter 16. The door 76 is preferably a hinged door wherein the hinge axis of the door 76 is parallel to the longitudinal axis 48 of the apparatus 10. A base 82 of the housing 12 has a convex surface that is at least partially locatable within a recess arranged at an end of the filter 16 to locate the filter 16 within a filter cavity defined by the housing 12.

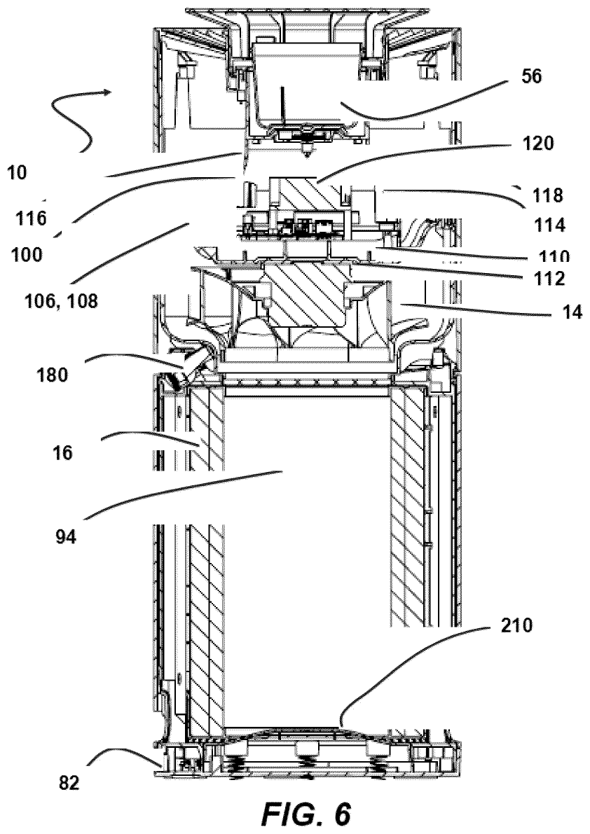

Referring to FIG. 7, the housing 12 defines the air passageway 28 between the air inlet structure 24 and the air outlet structure 26. The air passageway 28 generally directs air from the air inlet structure 24, through the filter 16 and fan 14, and toward the air outlet structure 26. The first portion of the filtered air 29a (i.e., the air that is drawn through the filter 16) exits the air outlet structure 26 which is provided in the form of the vent structure 32 including the plurality of vanes 50. The second portion of the filtered air 29b is directed within the headspace 34 of the receptacle 18 containing the treatment composition 56.

The housing 12 may be generally cylindrical and may be free standing. In one example, the overall size of the housing 12 is approximately 718 millimetres high with a diameter of approximately 287 millimetres and suited for filtering and cleaning rooms and dwellings. However, other shapes and support configuration are possible and contemplated herein such as square shapes and wall mounted variations. The apparatus 10 has a total weight of approximately 12 kilograms.

Referring to FIGS. 1 to 7, the housing 12 includes a first (upper) housing portion 78 coupled to a lower (second) housing portion 80. The lower housing portion 80 includes the base 82 supporting thereon the filter 16. The lower housing portion 80 includes an inner body 84 which extends from the base 82 defining the filter cavity 86. A wall of the inner body has an opening in a wall for receiving therein the filter 16. Outer wall structures 88 are attached to the inner body 84, wherein one of the outer wall structures 88 includes the hinged door 76 which aligns with an opening in the inner body 84 and is movable between the open and closable position to access, locate and replace the filter 16.

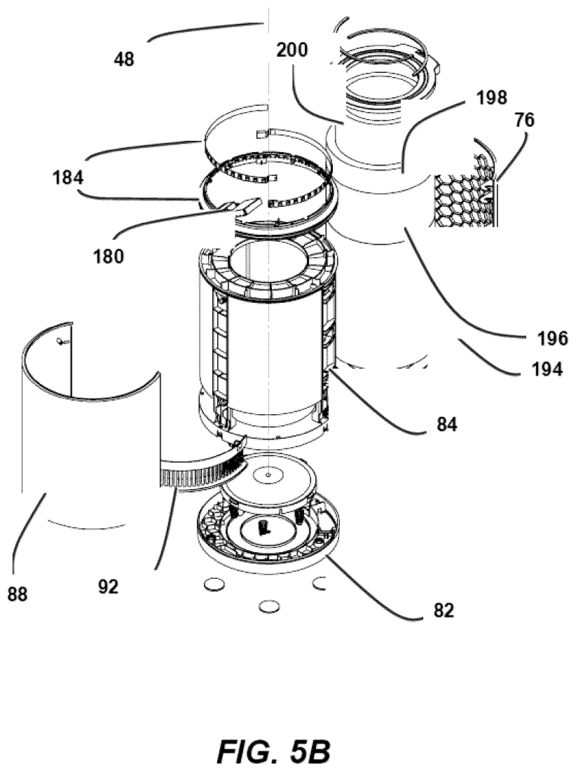

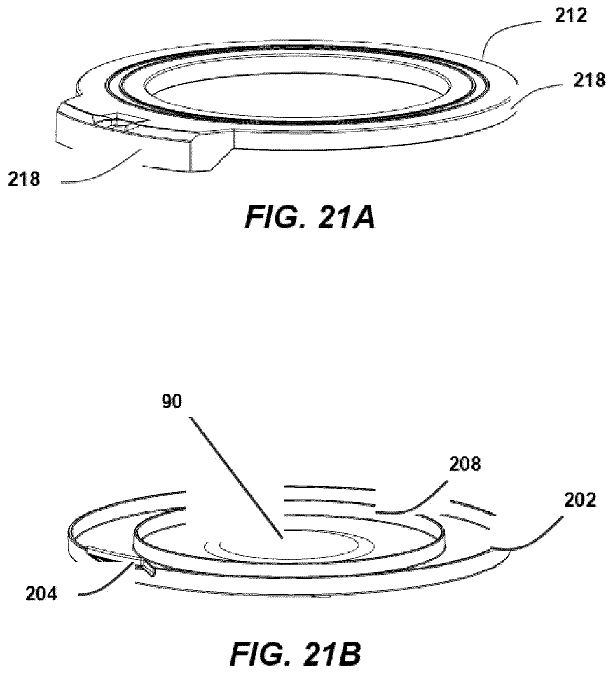

Referring to FIGS. 6 and 22, the base 82 of the housing 12 includes the convex, upturned base surface which has a profile which at least partially corresponds to a complimentary recess 90 on the bottom end surface of the filter 16 to align the filter 16 centrally within a filter cavity 86. Referring back to FIGS. 1 to 7, the inlet structure 24 of the apparatus 10 is provided in the form of an inlet vent which is located above the base 82 and circumferentially surrounds the lower portion of the lower housing portion 80. The inlet vent 24 can be provided in the form of a pair of inlet vent structures 92 which circumferentially surround the lower area of the lower housing portion 80 to draw ambient air within the passageway 28 of the apparatus 10 due to operation of the fan 14 located within the housing 12.

Ambient air that enters the lower housing portion 80 is drawn radially through the filter 16 and travels upwardly toward and through the fan 14. The filter 16 may be a multi-layered filter 16. The filter 16 has a central hole 94 which extends along and coaxial with the longitudinal axis 48 of the apparatus 10.

The upper housing portion 78 houses therein a lower inner housing section 96 coupled to an upper inner housing section 98. The lower inner housing section 96 houses therein the fan 14. The upper inner housing section 98 houses an inner shell housing 100, the controller 22 and the receptacle 18 having located therein the container 54 containing the treatment composition 56. The entrainment structure 20 is to coupled to an upper portion of the upper inner housing section 98. A cylindrical outer wall structure 102 is coupled to and surrounds the lower inner housing section 96 coupled to the upper inner housing section 98. The cylindrical outer wall structure 102 has a plurality of holes thereby allowing for a plurality of buttons 104 supported on an external wall of the upper inner housing section 98 to protrude therethrough or at least be accessed therethrough.

Referring to FIG. 6, a mounting structure 106 is mounted across to an inner surface of the lower inner housing section 96. The fan 14 is coupled to an underside of the mounting structure 106. The mounting structure 106 includes a plurality of arms 108 that extend outwardly from a plate portion 110 coupled to the fan 14. A grommet component 112 is located between the mounting structure 106 and an upper surface of the fan 14 to minimise transmission of vibrational forces.

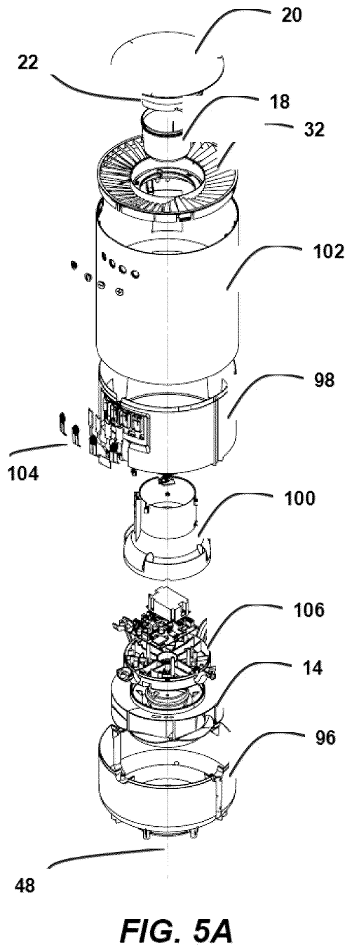

As described above and referring to FIGS. 5A, 5B, 6 and 7, the upper inner housing section 98 houses therein the inner shell 100. The inner shell 100 is supported upon the upper surface of the mounting structure 106. The inner shell 100 has a flared lower portion 114 that tapers toward an upper neck portion 116. The lower flared portion 114 houses the controller 22 which rests upon the plate portion 110 of the mounting structure 106. A midsection 118 of the inner shell 100 houses a power supply component 120 which is supported upon pins 122 which extend upwardly from the mounting structure 106 to thereby elevate the power supply component 120 above the controller 22.

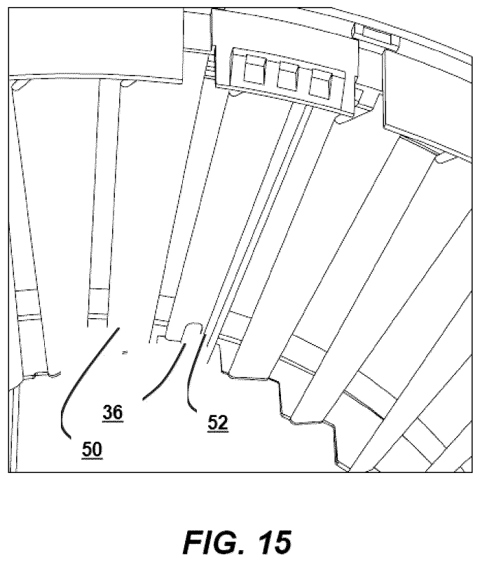

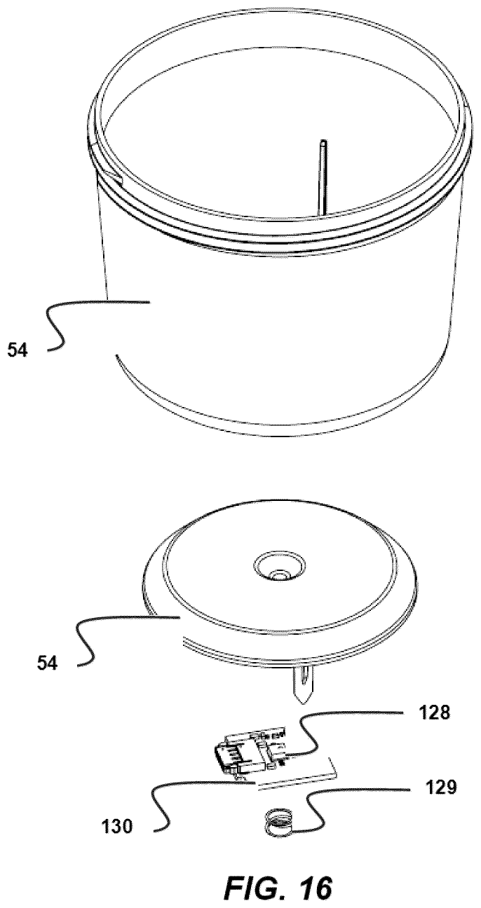

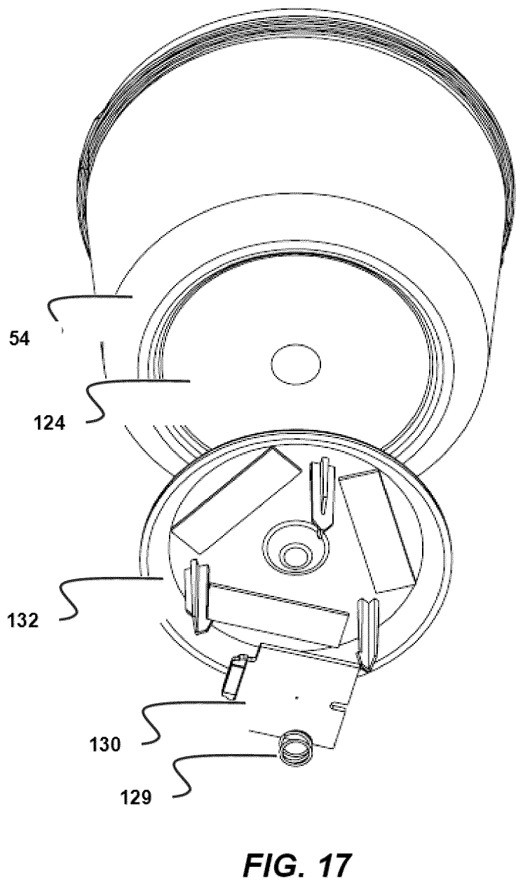

Referring to FIGS. 5 to 7 and 16 to 18, the receptacle 18 is arranged to provide the headspace 34 above the treatment composition 56 to allow for vapour 58 associated with the treatment composition 56 to build or accumulate within the headspace 34. The receptacle 18 containing the treatment composition 56 is located within the upper neck portion 116 of the inner shell 100. A base portion 124 of the receptacle 18 includes a central well 126 which has located therein a force sensor 128 and an associated printed circuit board (PCB) 130. A plunger 132 substantially encloses the force sensor 128 within the well 126. The plunger 132 is movable along the longitudinal axis 48 of the apparatus 10 under weight of the container 54 supported within the receptacle 18, wherein the weight of the container 54 is transferred to the plunger 132 which presses against the force sensor 128. The plunger 132 has a protrusion 134 extending from an underside surface which aligns with the force sensor 128. When no container 54 is located within the receptacle 18, the protrusion 134 of the plunger 132 is separated from the force sensor 128 such that the force sensor 128 does not sense the presence of the container 54. In this condition, the controller 22 does not operate the fan 14 based on the force signal (or lack thereof) indicative of insufficient weight present within the container 54 (or no container 54 at all). When the container 54 is located within the receptacle 18, the weight of the container 54 causes the plunger 132 to move along the longitudinal axis 48 such that the weight of the container 54 is transmitted by the plunger 132 against the force sensor 128, thereby detecting the presence of the container 54. If sufficient force is applied to the force sensor 128 by the container 54 containing the treatment composition 56, the fan 14 is operated by the controller 22. In a particular form, the weight is measured in 50-gram increments, wherein an alert can be output to the user by the apparatus 10 when 50 grams of treatment composition 56 remains, wherein the output can be via the light ring 184, and/or via the remote computing device 148. As shown in FIG. 16, a biasing structure 129, such as a spring, preferably provided in the form of a compression spring, supports the force sensor 128 provided by the PCB 130. If the container 54 is dropped into the device 10 when loading, the biasing structure 129 can absorb at least a portion of the impact force transferred to the force sensor 128, thereby reducing the likelihood of the force sensor 128 being damaged.

The composition 56 contained in the container 54 may be an evaporable vapour releasing composition such as gel or gel cartridge impregnated or formulated with essential oils or other suitable substances. The container 54 can be provided in the form of a removable cartridge adapted to hold the treatment composition 56, wherein the removable cartridge is located within the receptacle 18. The composition 56 is preferably supplied as a gel within the replaceable cartridge that is fitted, when installed, within the receptacle 18.

The replaceable cartridge 54 has a removable lid that is removed, and may be discarded, prior to the cartridge 54 being fitted to the receptacle 18. The fill level of the gel is such that the headspace 34 is maintained above the gel. However, it is noted that that replaceable cartridge 54 may also carry a liquid or viscous liquid. Other examples may omit the cartridge, wherein for example, a liquid or viscous liquid may be poured directly into the receptacle 18 26.

The composition 56 is preferably an evaporable substance or substance that emits a volatile adapted to affect, reduce, or kill air born pathological or biological material. An example of the composition 56 may be a gel insert, cartridge or disk that is impregnated with an essential off. The essential oils that have been found to affect, reduce, or kill mold and/or bacteria are, for example, clove oil, cinnamon, peppermint, rosemary, and eucalypt oils. Accordingly, a gel with a blend of clove oil, rosemary and eucalyptus extracts may be used. An example gel may include a polysaccharide gelling agent, or in other examples, a gelatin gelling agent. The gelling agent may be formed with a blend of evaporable oils.

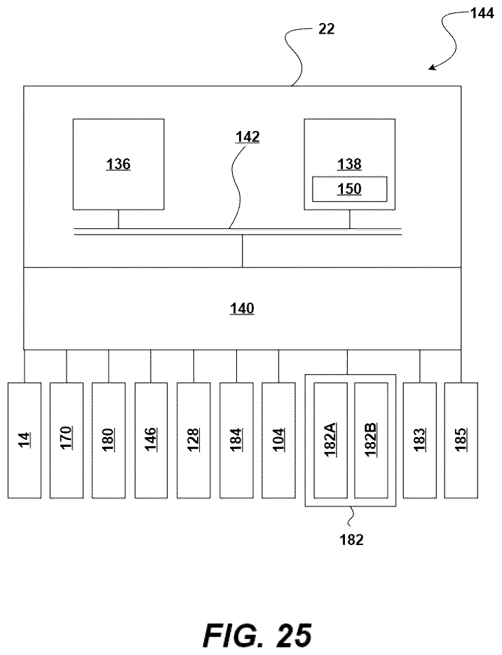

Referring to FIG. 25, the controller 22 of the air filtering and treatment apparatus 10 can include a processor 136, a memory 138, and an input/output (i/o) interface 140 coupled together via a bus 142. The memory 138 can include both volatile and non-volatile memory components. The controller 22, together with one or more peripheral devices coupled to the i/o interface 140, collectively form a control system 144 of the apparatus 10.

The controller 22 can include or be coupled to, via the i/o interface 140, a communication module 146. The communication module 146 can be a wireless communication module. In one form, the wireless communication module 146 can utilise wireless communication protocols such as Wi-Fi or Bluetooth, although other short-range or long-range communication protocols and modules can be utilised. The communication module 146 can be used to transfer and receive various data to and from a remote computing device 148.

The controller 22 can have stored in memory a computer program referred to as a control program 150. The control program 150 includes executable instructions which configure the controller 22 to perform various functions as described below.

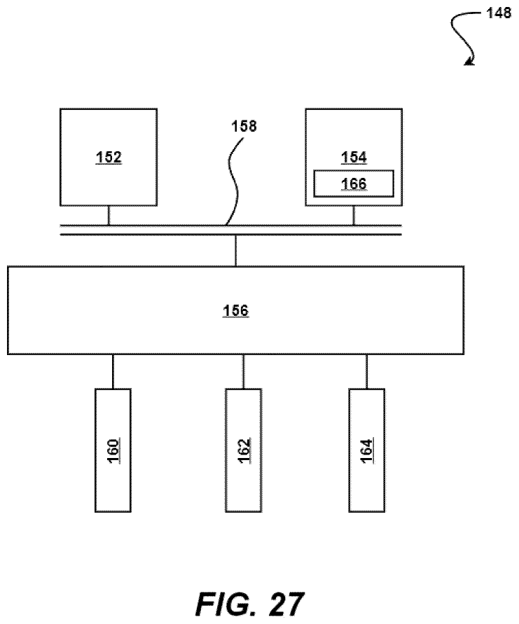

Referring to FIG. 27, the remote computing device 148 can include a processor 152, a memory 154, and an input/output interface 156, coupled together via a bus 158. The remote computing device 148 includes an input device 160 and an output device 162 which are coupled to the i/o interface. The remote computing device 148 also includes a communication module 164 which can be coupled to the i/o interface 156. As described above, the communications module 164 may include Wi-FI TM and or Bluetooth ™ functionality to allow communication with the apparatus 10. The remote computing device 148 may be a smart phone, tablet, laptop computer, desktop computer, or the like. In certain embodiments of the remote computing device 148, the input device 160 and output device 162 may be provided as an integral input/output device such as a touch screen interface.

The remote computing device 148 can have stored in the memory 154 a computer program 166 which is executable by the processor 152 of the remote computing device 148 to help facilitate communication with the air filtering and treatment apparatus 10 as well as provide output to the user. In certain embodiments, the computer program 166 can be an executable application, such as a smart phone application for instances where the remote computing device 148 is a smart phone or tablet device.

The controller 22 of the apparatus 10 may communicate, using the communication interface 140, directly or indirectly via a network 168 with the remote computing device 148. The remote computing device 148 may be used to configure the controller 22 to operate in particular modes and/or during particular operating times (Le., timer or on/off timer, fan 14 speed, entrainment positions, etc.,). The controller 22 may also be configured to send messages or alerts to the remote computing device 148.

The computer program 150 of the apparatus 10 or computer program 166 of the remote computing device 148 can configure the respective processor 136, 152 to extrapolate fan 14 speed and time of use, to determine the approximate life of both the HEPA filter 16 and composition/gel cartridge 54. Accordingly, methods performed by the controller 22 of the apparatus 10 and/or the remote computing device 148 may include various maintenance type routines. For example, the one or more computer programs 150, 166 may determine when the HEPA filter 16 or the composition requires changing and alert the user. For example, the controller 22 or the remote computing device 148 may time the on-time of the fan 14 and provide an alert when a predetermined number of hours have passed therefore being indicative of the life of the HEPA filter 16. The user may need to reset the time counter through the application software 166 of the remote computing device 148 once they have changed either HEPA filter 16 or container 54, although the newly sensed weight of the container 54 containing the composition can be used by the controller 22 to reset the counter automatically without user input or potentially only requiring confirmation in response to a notification output to the user.

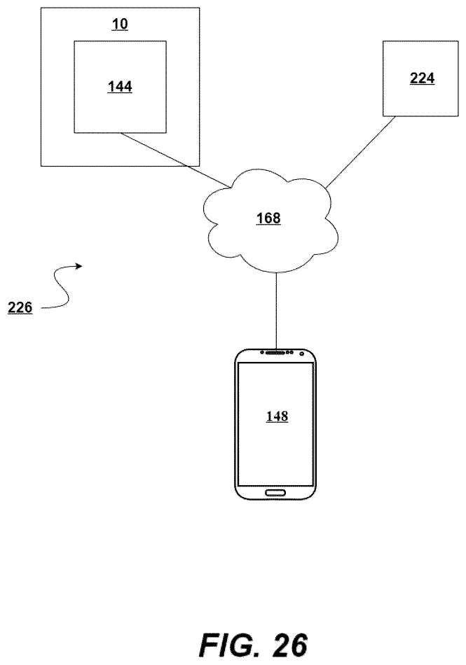

Referring to FIG. 26, the apparatus 10 may be configured to operate in an access point mode and/or Internet (client) mode. The access point mode implements a direct wireless connection between the apparatus 10 and the remote computing device 148. In the access point mode, the apparatus 10 can act as the network host and the remote computing device 148 can connect to the SSID of the apparatus 10 using a factory set password (which can later be changed via an application of application software operated by the smart device. While in this mode, the application software will only allow simple functions: a. Setting of the home/office Wi-Fi router credentials; b. Turning apparatus 10 ON or OFF c. Changing the user settings of the device such as fan 14 speed, timer, LED colour, etc.; d. Real-time reading of the air quality. In the Internet (client) mode, the apparatus 10 is connected online using the SSID and credentials of the local Wi-Fi hotspot (e.g. at home or office). These credentials would have been set up earlier when the apparatus 10 was in Access Point Mode. While in this mode, the apparatus 10 will log onto a cloud hosted web service on a server 224 of a system 226 where it can be uniquely identified, and its functions are made available over this service. The cloud hosted web service may allow a plurality of apparatus 10 to be simultaneously connected and controlled by a user. For example, a plurality of the apparatus 10 may provide dust and dew point readings at different locations within a dwelling or multiple dwellings. The cloud hosted web service may also allow related apparatuses 10, such as those owned by friends, connected, and share data such as air quality data. The cloud hosted web services may also allow full remote control of one or more of the apparatuses. In the Internet (client) mode, the apparatus 10 provides full functionality. These include all functions present in access point mode as well as other internet enabled functions: a. automatic resetting of the local device clock to internet time; b. ability to upload locally stored data related to air quality and usage; c. ability to be connected to a group of units at a home or office setting. For example, the user can allocate names to units such as ‘Master Bedroom’, ‘Living Room’ etc; d. set up of timed ON/OFF functions; and e. reporting of trend data from the cloud.

Referring to FIG. 25, the controller 22 of the apparatus 10 can have coupled thereto via the i/o interface 140 one or more sensors. The control program 150 of the controller 22 can use at least some of the sensor data to control the operation of the fan 14, in particular the speed of the fan 14, as well as facilitate output to the user requesting adjustment of the entrainment position of the entrainment structure 20.

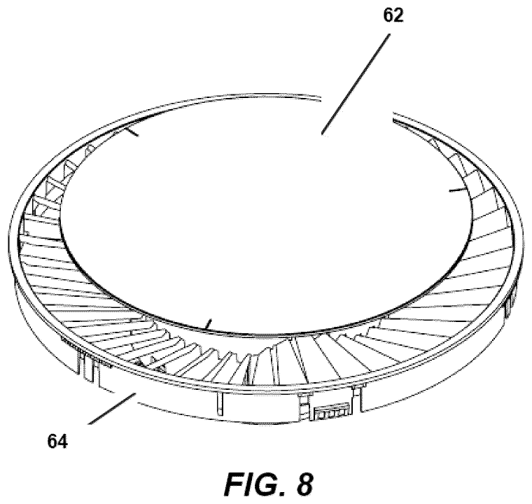

In one embodiment, the air filtering and treatment apparatus 10 can include a position sensor 170 which is in communication with the controller 22 via the i/o interface. The position sensor 170 is configured to sense a position of the entrainment structure 20 relative to the housing 12. The controller 22 is configured to receive a position signal from the position sensor 170 and facilitate feedback output of the current position of the entrainment structure 20 to the user. Referring to FIG. 9, in one form the position sensor 170 includes one or more photodetectors 172 (shown in broken line). The photodetectors 172 are configured to detect emitted light from one or more emitters 174 (shown in broken line). The one or more photodetectors 172 are configured to sense the presence or absence of light emitted by one or more emitters 174 to detect a position of the entrainment structure 20. In a specific form, the one or more photodetectors 172 are infrared photodetectors, and the one or more emitters 174 are infrared emitters. The photodetectors 172 and emitters 174 may be provided in an integrated emitter sensor module including a plurality of legs 176 extending from a body 178, wherein a first leg of the integrated emitter sensor includes a first photodetector and a first emitter, and a second leg of the integrated emitter sensor includes a second photodetector and a second emitter.

As described above, the controller 22 is configured to facilitate output of the current position of the entrainment structure 20 which can be achieved via the output device 162 of a remote computing device 148. Execution of the computer program 166 of the remote computing device 148 configures the processor 152 to receive, from the communication module 146 of the air filtering and treatment apparatus 10, position sensor 170 data indicative of the position of the entrainment structure 20 of the air filtering and treatment apparatus 10. The processor 152 of the remote computing device 148 is further configured to control an output device 162 of the remote computing device 148 to indicate to a user the sensed position of the entrainment structure 20. The output device 162 can be an electronic display of the remote computing device 148.

Referring to FIGS. 16 to 18 and 25, the air filtering and treatment apparatus 10 can include the force sensor 128 in communication with the controller 22. The force sensor 128 is configured to detect a presence of the container 54 containing the treatment composition 56 within the receptacle 18 and generate a force signal indicative of the presence of the container 54 within the receptacle 18. The controller 22 is configured to operate the fan 14 in response to receiving the force signal indicative of the presence of the container 54 within the receptacle 18. In one form, the force signal is indicative of an amount of force sensed by the force sensor 128. The controller 22 is configured to determine, based on the amount of force sensed by the force sensor 128, an amount of treatment composition 56 remaining in the container 54. The controller 22 is further configured to facilitate output, to the user, the amount of treatment composition 56 remaining in the container 54.

Facilitation of the output indicative of the amount of treatment composition 56 remaining in the container 54 can be performed via the remote computing device 148.

Execution of the computer program 166 by the remote computing device 148 configures the processor 152 to receive, from the communication module 164 of the apparatus 10, force sensor data indicative of the force sensed by the force sensor 128 and control the output device 162 of the remote computing device 148 to indicate to the user the amount of treatment composition 56 remaining in the container 54. The output device 162 can be an electronic display of the remote computing device 148.

Referring to FIGS. 6 and 25, the air filtering and treatment apparatus 10 can include a particle sensor such as an integrated particle sensing module 180 including a fan and particle sensor which is in electrical communication with the controller 22 via the i/o interface 140. The particle sensor is configured to sense a particle count of the ambient air drawn through the integrated sensing module by the fan 14 and facilitate output of the particle count to the user. Advantageously, the integrated nature of the module 180 allows for ambient air to be received and emitted via the same opening in the housing 12. The controller 22 can control activation of the fan 14 via the i/o interface 140 and receive a particle count signal from the sensor 180 via the i/o interface 140.

Facilitation of the output indicative of the particle count can be performed via the remote computing device 148. Execution of the computer program 166 by the remote computing device 148 configures the processor 152 to receive particle count data from the controller 22 of the apparatus 10 and control the output device 162 to present the particle count indicated by the particle count data to the user.

In one form, the controller 22 of the apparatus 10 is configured to compare the particle count to one or more particle count thresholds stored in memory 138. In response to the particle count equalling or exceeding one or more thresholds, the controller 22 can control an increase in the speed of the fan 14 and/or generate and transfer a notification to the remote computing device 148 instructing the user to alter the position of the entrainment structure 20 to increase an amount of filtered air to enter the headspace 34. In response to the particle count equalling or falling below one or more particle count thresholds, the controller 22 can control a decrease in the speed of the fan 14 and/or generate and transfer a notification to the remote computing device 148 instructing the user to alter the position of the entrainment structure 20 to decrease an amount of filtered air entering the headspace 34.

The one or more thresholds may include lower and upper particle counts defining one more operating mode ranges. In particular, the apparatus 10 can include three operating modes: off, low; and high. For each operating mode, the memory 138 may have an associated entrainment structure position together with the lower and upper particle count limits of the respective range. In response to receiving the particle count, the controller 22 can compare the sensed particle count to the defined ranges to determine whether the current operating mode matches the determined operating mode of the matching particle count range. If the current operating mode does not match the determined operating mode based on the particle count, the controller 22 can generate and transfer a notification requesting the user to alter the entrainment structure position. In addition, the fan speed may also be changed by the controller 22. In one form, upon the controller 22 receiving the position signal indicative of a change of the position of the entrainment structure 20, the controller 22 can determine a new fan 14 speed of the fan 14 based on the particle count and the current operating mode. For example, in response to the entrainment structure 20 being moved from the intermediate entrainment position to the maximum entrainment position, the fan 14 speed may be decreased due to the increased amount of the second portion of filtered air entering the headspace 34. Thus, the adjustment of the fan speed allows for fine micro-adjustment of the amount of ambient air being drawn into the apparatus 10 for filtering whilst the adjustment of the position of the entrainment structure 20 allows for macro-adjustments of the amount of the second portion of the filtered air entering the headspace 34.

Referring to FIG. 25, the air filtering and treatment apparatus 10 can also include dew point sensor 182 which is in communication with the controller 22 via the i/o interface 140. In some examples, the controller 22 may be configured to automatically activate the fan 14 of the apparatus 10 if detection of particles and the dew point exceeds a predetermined threshold or at least transfer a notification to the remote computing device 148. In a more specific example relating to the dew point sensor 182, this is a combined temperature sensor 182A and humidity sensor 183B to track conditions throughout the day and night where mould spores may be released. The controller 22 can have stored in memory 138 lookup table data defining one or more thresholds for activating the apparatus 10. In particular, the dew point is a function of temperature and relative humidity. For example, al 20 °C at 50% relative humidity, the dew point is about 9 °C. Mould may start to grow at room humidity of 80%. This means that in the given example, mould may start to grow at a surface temperature of about 13 °C or less on the walls. Accordingly, for example, the apparatus 10 may begin to operate in an active operating mode or generate and transfer a notification to the remote computing device 148 indicative of the detection. Should conditions for mould spore release occur, the user can be alerted and/or the apparatus 10 may automatically switch on the fan 14 (depending on user settings). The hardware for the sensor 182 could be either an off-the-shelf dew point sensor 182, or individual temperature/humidity sensors—whichever is deemed suitable.

Whilst examples have been described above where the controller 22 facilitates output to the user via an output device 162 of a remote computing device, in additional or alternate configurations, the controller 22 can facilitate output to the user by operating a local output device 162 which is in communication with the i/o interface 156 of the controller 22. In one form, the output device 162 can be provided in the form of one or more light emitting diodes. In a specific form, the one or more lighting emitting diodes can be provided in the form of a light ring 184, as shown in FIGS. 5 and 25, that is mounted within the midsection of the housing 12. The light ring 184 is configured to support lights in a ring structure. The lights may emit a red, green, and/or blue light. The light ring 184 is in electrical communication with the i/o interface, wherein the controller 22 can control the light ring 184 to emit a particular colour light or a particular lighting pattern or sequence, such as flashing or blinking, to provide output to the user. In one form, the light ring 184 of the apparatus 10 is configured to change colour to indicate various statuses of the apparatus 10. For example, the light ring 184 may emit: a solid white light to indicate that the apparatus 10 is switched on; a solid orange light to indicate that the filter 16 requires changing; a blinking orange light to indicate that the gel/composition requires changing; a solid red light to indicate that both the HEPA and gel/composition require changing.

A shown in FIGS. 1 to 7, the air filtering and treatment apparatus 10 can further include a plurality of input buttons 104 which is operatively coupled to the i/o interface 140 of the controller 22. In particular, the air filtering and treatment apparatus 10 can include four buttons, namely a power button 186, an increase fan speed button 188, a decrease fan speed button 190, and a configurable button 192. More specifically, the configurable button 192 is a sleep mode button to operate the air filtering and treatment apparatus 10 during a period when the user is sleeping.

In one form, the user can set a customised setting of the air filtering and treatment apparatus 10 during the sleep mode. In particular, the controller 22 is configured to receive customised setting data indicative of a customised setting for operating the apparatus 10.

The controller 22 is configured to store, in memory 138, an association between the customised setting and the button 192. The controller 22 is then configured to receive a signal indicative of the button being selected by the user. Finally, the controller 22 is configured to operate the fan 14 according to the customised setting stored in the memory. In one form, the user can set a fan 14 speed of the of the fan 14 during the customised sleep mode.

In one form, the computer program 166 executable by the remote computing device 148 can allow the user to select the same functionality via the user interface of the computer program 166. For example, the user interface of the computer program 166 may present a plurality of soft buttons which the user can select using an input device of the remote computing device 148 to effectively perform the same function as selecting one or more of the physical buttons provided on the apparatus 10. Data indicative of the selected soft button can be transferred via the communication module of the remote computing device to the controller 22 of the apparatus 10 to effectively indicate that one of the corresponding physical buttons has been selected such that the controller 22 performs the associated operation accordingly.

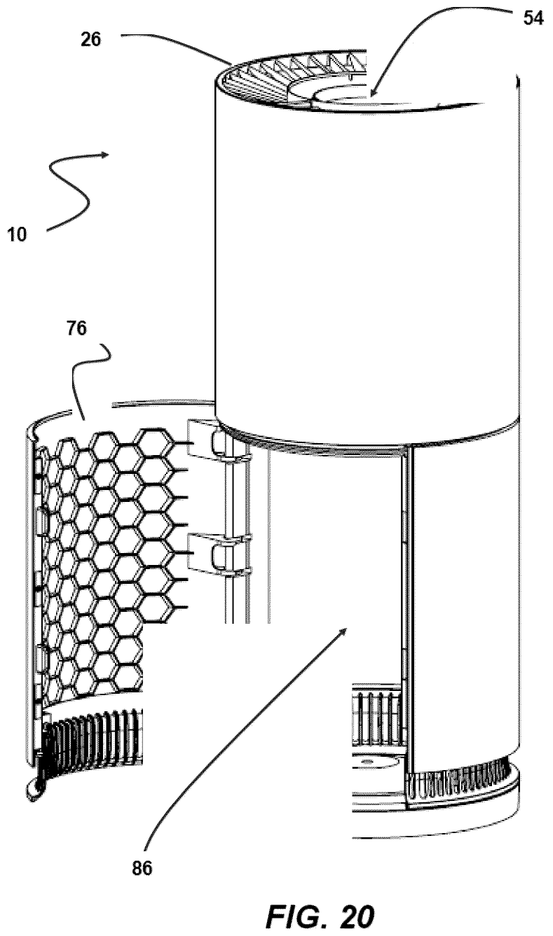

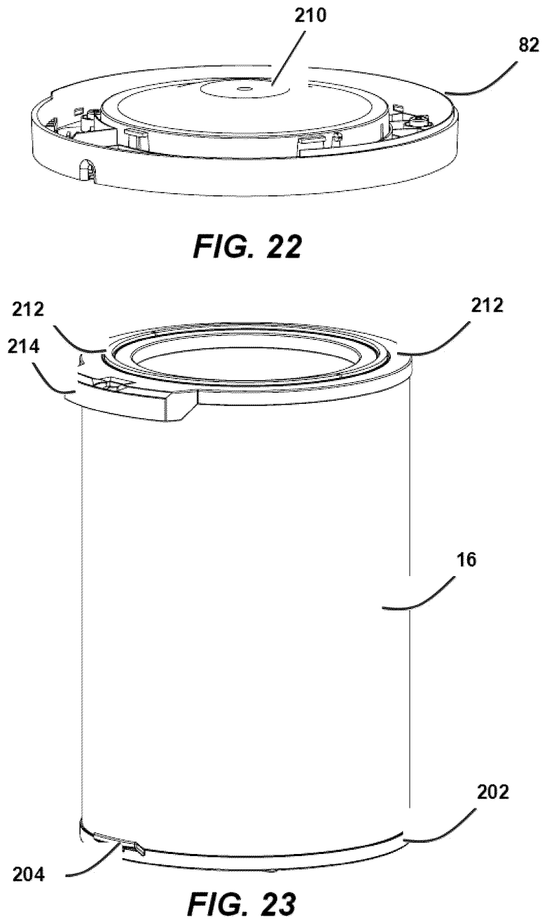

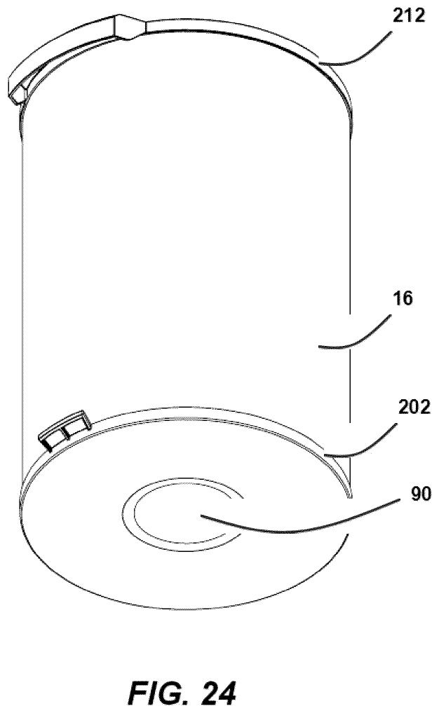

Turning now to FIGS. 5, 21A, 21B, and 22 to 24, there is shown the filter 16 of the apparatus 10 or parts thereof, wherein the filter 16 is arranged to filter air passing through the air passageway 28. Ambient air is drawn through the filter 16 such that the filtered air 28 leaves the filter 16 and travels toward and through the fan 14. The filter 16 is preferably a multi-layered filter. In order of the most outer layer to the most inner layer, the filter 16 includes a mesh screen layer 194, a HEPA filter layer 196, a sanitising layer 198, and an active carbon layer 200. The HEPA filter layer 196 may be capable of filtering to 1.0 particle size.

The filter 16 includes a base cap 202 that is supported upon the base 82 of the housing 12. The filter layers 194-200 are supported upon an upper layer of the base cap 202 of the filter 16. The base cap 202 includes a base handle 204 extending from the perimeter thereof. The base cap 202 has a raised edge 206 around the perimeter to support the external surface of the filter layers 194-200. A raised wall 208 extends upwardly from the base cap 202 midway between the perimeter and the centre of the cap 202. The further raised circular wall 208 supports an inner wall of the filter layers 194-200, thereby defining a central void for the filtered air to be drawn upward through the fan 14. The central portion of the base cap 202 includes a raised section 210 defining the recess 90 which the raised central portion 210 of the base 82 rests at least partially therein.

The base 82 of the housing 12 includes an upper base portion 82A that is supported by a lower base portion 82B. The upper base portion 82A is supported within a cavity of the lower base portion 82B. Located within the cavity are one or more biasing components such as one or more springs (not shown) and a filter sensor 183 in communication with the controller 22 via the i/o interface 140. The filter sensor 183 can be a switch which has a first state when the upper base portion 82A has no filter 16 supported thereon and a second state when the upper base portion 82A has a filter 16 supported thereon. As such, force which is applied to the upper base portion 82A by the user inserting the filter 16 or the filter 16 being supported within the housing 12 causes the upper base portion 82A to move downwards and towards the lower base portion 82B whilst the one or more springs are compressed. This movement causes the switch 183 to transition from the first state to the second state, thereby indicating t the controller 22 that a filter 16 is present within the apparatus 10. When the filter 16 is removed, the one or more springs bias the upper base portion 82A away from the lower base portion 82B such that the switch 183 transitions from the second state to the first state thereby indicating to the controller 22 that the filter 16 has been removed. The controller 22 is configured to determine if the filter 16 is present within the apparatus 10 based on a filter sensor signal generated by the filter sensor 183 received from the filter sensor 183. If the filter sensor signal indicates that the filter 16 is not contained within the housing 12, the controller 22 will disable the operation of the fan 14, but in the event that the filter sensor signal indicates that the filter 16 is present within the housing 12, the controller 22 enables operation of the fan 14 accordingly.

At an opposing end of the filter 16 relative to the base cap 202 is a head cover 212. The head cover 212 includes a substantially annular profile. A top handle 214 extends from the external perimeter of the head cover 212 which aligns with the base handle 204 in a parallel manner with the longitudinal axis 48 of the apparatus 10. The head cover 212 has a central hole 216 which aligns with the central hole 94 located within the filter layers 194-200. Like the base cap 202, the outer perimeter of the head cover 212 includes an outer wall 218 extending downwardly which supports the external wall of the filter layers 194-200, and an inner wall 220 defining the central hole 216 which supports an inner surface of the filter layers 194-200. A base surface of the lower housing portion 80 includes an inlet vent 220 which fits within the central hole 216 of the head cover 212. A seal 222 is located between the upper surface of the head cover 212 and the inlet vent 220 of the lower housing portion 80. The seal 222 is configured to prevent bypass of airflow.

The apparatus 10 may include a door sensor 185 to sense an open or closed position of the door 76. The door sensor 185 may be provided in the form of a switch having a first state and a second state. The door sensor 185 is in communication with the controller via the i/o interface When the door 76 is closed, the door sensor 185 is in the first state, wherein a door sensor signal is transferred from the door sensor 185 to the controller 22, wherein the controller 22 enables operation of the fan 16 (insofar as that other signals do not indicate that the fan 16 should be disabled). When the door 76 is open, the door sensor 185 transitions to the second state, wherein the door sensor signal is transferred from the door sensor 185 to the controller 22, wherein the controller 22 disables the operation of the fan 16 until the door 76 is closed.

Referring to FIGS. 5 to 7, the fan 14 may be an electric axial fan 14 arranged to rotate about the longitudinal axis 48 of the apparatus 10. The air flow is therefore generally vertical between the inlet structure 24 and the outlet structure 26. As previously described, the fan 14 is controlled by the controller 22 located in the inner shell 100. The fan 14 is supported by the mounting structure 106.

In operative use, air is drawn through the housing 12 using the fan 14 such that the first portion of filtered air exits the outlet structure 26, and the second portion of filtered air exits the entrainment outlets 30 having entrained therewith vapour 58 of the treatment composition 56. The ambient air drawn with the housing 12 is filtered using the filter 16 arranged between inlet structure 24 and the outlet structure 26. Sensor measurements, such as a measurement of a particle count, may be used by the controller 22 to adjust the fan 14 speed and/or facilitate output to the user an instruction to adjust a position of the entrainment structure 20. For example, sensor data indicalive of the particle count may be used by the controller 22 to transfer a notification to the remote computing device 148. Based on the output provided to the user, the user can manually rotate the entrainment structure 20 relative to the housing 12 to adjust the entrainment position of the entrainment structure 20. The controller 22 continues to sample and monitor the one or more sensor measurements such that the user may be provided various instructions over time to adjust the position of the entrainment structure 20.

In certain forms, the apparatus 10 has a selectable operable flow rate ranging from about 100 m³/h to about 450m³/h. In certain forms, the apparatus 10 can additionally include a “turbo” operable flow rate of about 500m³/h. In a preferable form, the fan can include 10 operating speeds.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps

The reference in this specification to any known matter or any prior publication is not, and should not be taken to be, an acknowledgment or admission or suggestion that the known matter or prior art publication forms part of the common general knowledge in the field to which this specification relates.

While specific examples of the invention have been described, it will be understood that the invention extends to alternative combinations of the features disclosed or evident from the disclosure provided herein.

Many and various modifications will be apparent to those skilled in the art without departing from the scope of the invention disclosed or evident from the disclosure provided herein.

### LIST OF INTEGERS

air filtering and treatment apparatus 10
housing 12
fan 14
filter 16
receptacle 18
entrainment structure 20
controller 22
air inlet structure 24
air outlet structure 26
air passageway 28
filtered air 29
first portion of filtered air 29a
second portion of filtered air 29b
entrainment outlets 30
vent structure 32
headspace 34
inlet apertures 36
entrainment inlets 38
channel 40
biasing component 42
stepped recesses 44
protrusion 46
longitudinal axis 48
vanes 50
deflecting member 52
container 54
treatment composition 56
vapour 58
wall of entrainment structure 60
first portion of the entrainment structure 62
second portion of the entrainment structure 64
ribs 68
neck of entrainment structure 70
first entrainment inlet 72
second entrainment inlet 74
door 76
upper housing portion 78
lower housing portion 80
base 82
upper base portion 82A
lower base portion 82B
inner body 84
filter cavity 86
outer wall structures 88
recess on end of filter 90
pair of inlet vent structure 92
central hole of filter 94
lower inner housing section 96
upper inner housing section 98
inner shell 100
outer wall structure 102
buttons 104
mounting structure 106
arms 108
plate portion 110
grommet component 112
flared lower portion 114
upper neck portion 116
midsection 118
power supply component 120
pins 122
base portion of receptacle 124
well 126
force sensor 128
biasing structure 129
force sensor PCB 130
plunger 132
protrusion of plunger 134
processor of controller 136
memory of controller 138
i/o interface of controller 140
bus of controller 142
control system 144
communication module 146
remote computing device 148
control program 150
processor of remote computing device 152
memory of remote computing device 154
input/output (i/o) interface of remote computing device 156
bus of remote computing device 158
input device of remote computing device 160
output device of remote computing device 162
communication module of remote computing device 164
computer program of remote computing device 166
network 168
position sensor 170
photodetectors 172
emitters 174
legs 176
body 178
integrated particle sensing module 180
dew point sensor 182
temperature sensor 183
light ring 184
temperature sensor 185
power button 186
increase fan speed button 188
decrease fan speed button 190
configurable button 192
mesh screen layer 194
a HEPA filter layer 196
a sanitising layer 198
an active carbon layer 200
base cap 202
base handle 204
outer edge of base cap 206
inner wall of base cap 208
raised central portion 210
head cover 212
top handle 214
central hole of head cover 216
outer wall of head cover 218
inlet vent of lower housing portion 220
seal 222
server 224
system 226

1. An air filtering and treatment apparatus including:
a housing having an air inlet structure configured to receive ambient air, an air outlet structure configured to release a first portion of filtered air, and an air passageway between the air inlet structure and the air outlet structure;
a fan arranged to urge air through the air passageway between the air inlet structure and the air outlet structure;
a controller configured to control the fan;
a filter arranged to filter air passing through the air passageway;
a receptacle located toward the air outlet structure relative to the filter having located therein a treatment composition, wherein an evaporable vapour associated with the treatment composition is able to accumulate in a headspace of the receptacle; and
an entrainment structure, in fluid communication with the air passageway, having one or more entrainment outlets for releasing a second portion of filtered air, wherein the entrainment structure is selectively movable relative to the housing to control an amount of the evaporable vapour entrained with the second portion of filtered air exiting the one or more entrainment outlets.

2. The air filtering and treatment apparatus according to claim 1, wherein the housing includes a vent structure which is coupled and rotatable relative to the entrainment structure, wherein the entrainment structure is configured to rotate relative to the vent structure to adjust an amount of the second portion of the filtered air entering the headspace to thereby control the amount of the evaporable vapour entrained with the second portion of filtered air exiting the one or more entrainment outlets.

3. The air filtering and treatment apparatus according to claim 2, wherein the vent structure includes a plurality of inlet apertures in fluid communication with the headspace which enable the second portion of filtered air to be directed within the headspace, wherein the entrainment structure includes a wall having one or more gaps, wherein the entrainment structure is configured to rotate relative to the vent structure to adjust alignment of the one or more gaps with the plurality of inlet apertures to thereby control an amount of the evaporable vapour entrained with the second portion of filtered air exiting the one or more entrainment outlets.

4. The air filtering and treatment apparatus according to claim 3, wherein the vent structure includes a channel housing a biasing component therein and a plurality of stepped recesses, wherein a neck portion of the entrainment structure has a protrusion which is receivable within, movable along and biased, by the biasing component, into engagement with one of the stepped recesses, wherein a force applied by the user to the entrainment structure along a longitudinal axis of the apparatus disengages the protrusion from a respective stepped recess thereby allowing the entrainment structure to be rotated relative the housing and allow the protrusion to engage with a different stepped recess to thereby control the amount of the evaporable vapour entrained with the second portion of the filtered air exiting the one or more entrainment outlets.

5. The air filtering and treatment apparatus according to claim 3, wherein each inlet aperture is diametrically offset relative to a remainder of the inlet apertures.

6. The air filtering and treatment apparatus according to claim 2, wherein the entrainment structure is configured to substantially enclose the receptacle within the housing, wherein the entrainment structure is detachable from the vent structure of the housing to allow access to the receptacle.

7. The air filtering and treatment apparatus according to claim 1, further including a position sensor, in communication with the controller, configured to sense a position of the entrainment structure relative to the housing, wherein the controller is configured to receive a position signal from the position sensor and facilitate output of the position of the entrainment assembly to the user.

8. The air filtering and treatment apparatus according to claim 7, wherein the position sensor includes one or more photodetectors, wherein the air filtering and treatment apparatus includes one or more emitters, wherein the one or more photodetectors are configured to sense the presence or absence of light emitted by one or more emitters to detect a position of the entrainment assembly.

9. The air filtering and treatment apparatus according to claim 8, wherein the one or more photodetectors are infrared photodetectors, and the one or more emitters are infrared emitters.

10. The air filtering and treatment apparatus according to claim 1, wherein the air outlet structure includes a plurality of vanes, wherein at least some of the vanes have extending therefrom a deflecting member to deflect the second portion of the filtered air radially inward within the headspace of the receptacle such that the evaporable vapour is entrained with the second portion of filtered air when exiting the one or more entrainment outlets.

11. The air filtering and treatment apparatus according to claim 10, wherein each deflecting member extends substantially orthogonally from the respective vane.

12. The air filtering and treatment apparatus according to claim 1, further including a force sensor in communication with the controller, wherein the force sensor is configured to detect a presence of a container containing the treatment composition within the receptacle and generate a force signal indicative of the presence of the container within the receptacle, wherein the controller is configured to operate the fan in response to receiving the force signal indicative of the presence of the container within the receptacle.

13. The air filtering and treatment apparatus according to claim 12, wherein the force signal is indicative of an amount of force sensed by the force sensor, wherein the controller is configured to determine, based on the amount of force sensed by the force sensor, an amount of treatment composition remaining in the container and facilitate output, to the user, the amount of treatment composition remaining in the container.

14. The air filtering and treatment apparatus according to claim 1, further including an integrated sensing module including a fan and particle sensor, wherein the particle sensor is configured to sense a particle count of the ambient air drawn through the integrated sensing module by the fan and facilitate output of the particle count to the user.

15. The air filtering and treatment apparatus according to claim 1, wherein the housing includes a door movable between an open position and closed position to access, locate and replace the filter.

16. The air filtering and treatment apparatus according to claim 15, wherein the base has a convex surface that at least partially locatable within a recess arranged at an end of the filter to locate the filter within the housing.

17. The air filtering and treatment apparatus according to claim 1, further including an input button to operate the apparatus according to a customized setting, wherein the controller is configured to:
receive customized setting data indicative of a customized setting for operating the apparatus;
store, in memory, an association between the customized setting and the button;
receive a signal indicative of the button being selected by the user; and
operate the fan according to the customized setting stored in the memory.

18. An air filtering and treatment apparatus including:
a housing having an air inlet structure configured to receive ambient air, an air outlet structure configured to release a first portion of filtered air, and an air passageway between the air inlet structure and the air outlet structure;
a fan arranged to urge air through the air passageway between the air inlet structure and the air outlet structure;
a controller configured to control the fan;
a filter arranged to filter air passing through the air passageway;
a receptacle located toward the air outlet structure relative to the filter having located therein a treatment composition, wherein an evaporable vapour associated with the treatment composition is able to accumulate in a headspace of the receptacle;
an entrainment structure, in fluid communication with the air passageway, having one or more entrainment outlets for releasing a second portion of filtered air, wherein the entrainment structure is selectively movable relative to the housing to control an amount of the evaporable vapour entrained with the second portion of filtered air exiting the one or more entrainment outlets, wherein the housing includes a vent structure which is coupled and rotatable relative to the entrainment structure, wherein the entrainment structure is configured to rotate relative to the vent structure to adjust an amount of the second portion of the filtered air entering the headspace to thereby control the amount of the evaporable; vapour entrained with the second portion of filtered air exiting the one or more entrainment outlets, wherein the entrainment structure is configured to substantially enclose the receptacle within the housing, wherein the entrainment structure is detachable from the vent structure of the housing to allow access to the receptacle;
a communication module; and
a computer program executable by a processor of a remote computing device, wherein execution of the computer program by the processor configures the processor to:
receive, from the communication module of the air filtering and treatment apparatus, position sensor data indicative of the position of the entrainment structure of the air filtering and treatment apparatus; and
control an output device of the remote computing device to indicate to a user the sensed position of the entrainment assembly.

19. An air filtering and treatment apparatus including:
a housing having an air inlet structure configured to receive ambient air, an air outlet structure configured to release a first portion of filtered air, and an air passageway between the air inlet structure and the air outlet structure;
a fan arranged to urge air through the air passageway between the air inlet structure and the air outlet structure;
a controller configured to control the fan;
a filter arranged to filter air passing through the air passageway;
a receptacle located toward the air outlet structure relative to the filter having located therein a treatment composition, wherein an evaporable vapour associated with the treatment composition is able to accumulate in a headspace of the receptacle;
an entrainment structure, in fluid communication with the air passageway, having one or more entrainment outlets for releasing a second portion of filtered air, wherein the entrainment structure is selectively movable relative to the housing to control an amount of the evaporable vapour entrained with the second portion of filtered air exiting the one or more entrainment outlets; and
a force sensor in communication with the controller, wherein the force sensor is configured to detect a presence of a container containing the treatment composition within the receptacle and generate a force signal indicative of the presence of the container within the receptacle, wherein the controller is configured to operate the fan in response to receiving the force signal indicative of the presence of the container within the receptacle, wherein the force signal is indicative of an amount of force sensed by the force sensor, wherein the controller is configured to determine, based on the amount of force sensed by the force sensor, an amount of treatment composition remaining in the container and facilitate output, to the user, the amount of treatment composition remaining in the container; and
a computer program executable by a processor of a remote computing device, wherein execution of the computer program by the processor configures the processor to:
receive, from the communication module, force sensor data indicative of the force sensed by the force sensor; and
control an output device of the remote computing device to indicate to the user the amount of treatment composition remaining in the container.

20. The An air filtering and treatment apparatus of claim 18, further including:
an input button to operate the apparatus according to a customised setting, wherein the controller is configured to:
receive customised setting data indicative of a customised setting for operating the apparatus;
store, in memory, an association between the customised setting and the button;
receive a signal indicative of the button being selected by the user; and
operate the fan according to the customised setting stored in the memory;
wherein the execution of the computer program by the processor further configures the processor to:
receive customised setting data from the user via the remote computing device; and
transfer, to the communication module of the air filtering and treatment apparatus, the customised setting data for storing in memory in association with the button.
